(19) United States
(12) Patent Application Publication
(10) Pub. No.: US 2008/0305160 A1
(43) Pub. Date: Dec. 11, 2008

(54) Oral Medicament For The Modified Release Of At Least One Active Principle, In Multimicrocapsule Form

(75) Inventors: Florence Guimberteau, Montussan (FR); Catherine Castan, Orlienas (FR); Remi Meyrueix, Lyon (FR); Gerard Soula, Meyzieu (FR)

Correspondence Address: PATTON BOGGS LLP, 8484 WESTPARK DRIVE, SUITE 900, MCLEAN, VA, 22102, US

(73) Assignee: Flamel Technologies, Venissieux (FR)

(21) Appl. No.: 11/791,466

(22) PCT Filed: Nov. 2, 2005
(86) PCT No.: PCT/FR2005/050922

§ 371 Date: Jan. 8, 2008

(30) Foreign Application Priority Data
Nov. 24, 2004 (FR) 0452748

(51) Int. Cl. A61K 9/52; A61K 9/26; A61K 31/585; A61K 31/4166; A61K 31/435; A61K 31/43; A61K 31/522

(52) U.S. Cl. 424/456; 424/490; 424/497; 424/494; 424/498; 424/469; 514/173; 514/390

(57) ABSTRACT

The field of the invention is that of oral medicaments or pharmaceutical compositions, in particular of the type including one or more active principles. The aim of the invention is to provide an improved oral medicament to be administered in one or several daily doses and enabling the modified release of active principles (in particular of one active principle), whereby the prophylactic and therapeutic effectiveness of said medicament is improved. This aim is achieved by the oral multimicrocapsule galenic form according to the invention, in which the active principle release is controlled by a dual release trigger mechanism: “time-dependent trigger” and “pH-dependent trigger”. Said medicament includes microcapsules providing the modified release of the active principle, each comprising a core containing

### FIELD OF THE INVENTION

The field of the present invention is that of micro-particulate systems for the delayed and controlled release of active principle(s) AP(s), for oral administration.

The APs envisioned in the present invention are in particular those which have an absorption essentially limited to the upper parts of the gastrointestinal tract, located upstream of the colon (of the ileocecal junction), and which represent a large majority of pharmaceutical active principles. The active principles most especially targeted are “low-solubility” active principles.

More specifically, the invention relates to a microparticulate galenic form for delayed and controlled release, for which the controlled release phase is triggered in a definite manner by means of a double mechanism: “time-dependent” release triggered after a certain period of time spent in the stomach and “pH-dependent” release triggered by a change in pH when the particles enter the small intestine and which begins without any lag time. The microparticles of the present invention are microcapsules containing at least one active principle (AP)— with the exclusion of carvedilol—, having a particle size of less than 2000 microns, individually coated with a coating film for the delayed and controlled release of the AP.

The invention also relates to the microcapsules for the modified release of at least one active principle, taken as such.

In the present disclosure, the expression “low-solubility active principle” denotes any active principle, with the exclusion of carvedilol, which has a solubility of less than or equal to approximately 50 g/l, preferably less than or equal to approximately 20 g/l, even more preferably less than or equal to approximately 5 g/l and, for example, less than or equal to 0.1 g/l.

In the present disclosure, the term “microcapsules” denotes microparticles of active principle, coated with at least one coating for the modified release of at least one active principle (in particular, a low-solubility active principle).

In the present disclosure, the term “carvedilol” denotes carvedilol per se, one or more pharmaceutically acceptable salts of carvedilol or one or more pharmaceutically acceptable esters of carvedilol, or any mixture of these active agents.

In the present disclosure, the expression “modified release” denotes, without distinction, a prolonged release of the active principle(s), beginning as soon as the microcapsules are brought into contact with the dissolving medium and extending from 0.5 h to 24 h, preferably 1 h to 10 h, or a release of the active principle(s) that begins only after a predetermined period (lag time) ranging, for example, from 0.5 to several hours, with a release time for 50% of the active principle(s) which is typically several hours and which can extend from 0.5 to 30 hours, for example.

In the present disclosure, the expression “immediate release” denotes a release of the active principle(s) that begins as soon as the galenic form is brought into contact with the dissolving medium (in vivo or in vitro) with a release time for 80% of the active principle(s) which is less than or equal to 1 h, and for example less than or equal to 20 min.

Systems for the delayed and controlled release of active principle(s) are particularly useful when it is desirable, for chronobiological reasons, for the active principle(s) to be “bioabsorbed” at a specific time of the day in order to be in phase with the circadian cycle. It may, for example, be advantageous for the active principle(s) to be bioabsorbed very early in the morning in order to ensure therapeutic cover when the patient wakes up, without however restricting the latter to an early wake-up. To do this, the galenic system ingested by the patient, for example in the evening after the evening meal, should allow a delayed release of the active principle(s).

In the knowledge that another obligatory rule for the specialist in galenics is to guarantee that the medicament prescribed will be absorbed by the patient, it is essential, in the case of a delayed-release form, to have a complete guarantee of release of the active principle at a given moment so as to obtain the therapeutic effect. Now, it must be noted that the delayed-release forms that existed up until recently could not definitely ensure the release of the AP in a stipulated time period, which can be vital for the patient in certain pathologies, for instance that of cardiovascular diseases.

Another desired property for systems for the delayed and controlled release of active principle(s) is an improvement in the plasma concentration profile obtained after administration. The intended aim is to obtain a plasma profile which is maintained above the effective therapeutic concentration, for as long as possible, in order to maximize the duration of action of the active principle(s), and therefore its (their) therapeutic effectiveness. This aim comes up against the residence time of the active principle(s) in the blood compartment, which is most commonly much less than one day. In order to achieve this aim, it would therefore be advisable to prolong the bioabsorption time of the active principle(s) AP(s) by judicious adjustment of the release of the active principle(s) in front of its (their) bioabsorption window, in the upper parts of the gastrointestinal tract.

Various forms for the modified release of active principle(s) have been developed in order to attempt to solve the abovementioned problems of chronotherapy and of maintenance of a high plasma profile for as long as possible.

pH-dependent delayed-release forms are thus known, which are obtained by coating the active principle(s) by means of a layer of enteric polymer, for example of copolymer of methacrylic acid and methacrylic acid methyl ester: EUDRAGIT® L. This type of enteric coating is known to provide a reduced permeability under the acidic pH conditions of the stomach and to dissolve when the pH goes back up to a value close to that which occurs in the small intestine, thus releasing the active principle(s). However, intra- and inter-individual variability in gastric pH conditions and in the duration of gastric emptying do not make it possible to definitely ensure the release of the active principle(s) after a given period of time.

Delayed-release systems that are purely dependent on the time after ingestion (“time-dependent”), i.e. systems for which the release of the active principle(s) is triggered after a given period of time spent in the gastrointestinal tract, are, moreover, known and are not satisfactory either. In fact, due to the intra- and interindividual variability in gastric residence time, the release of the active principle(s) can take place after the latter has passed in front of its absorption window, which is located, for most active principles, in the upper parts of the gastrointestinal tract. The bioabsorption can thus be very low, or even zero.

However, it wasn't until the multimicroparticulate galenic system as disclosed in PCT patent application WO-A-03/030878 that very significant progress was obtained, in particular with regard to the abovementioned problems of chronotherapy and of maintenance of a high plasma profile for as long as possible. This system for the delayed, controlled and definite release of the active principle(s) is characterized by a dual mechanism for triggering the release of the active principle(s): “time-dependent” release triggered after a controlled period in the stomach, without any change in pH, and “pH-dependent” release triggered by a raised pH, when the galenic form penetrates the intestine. These two triggering factors for the release of the active principle(s) are placed in series and confer very safe use on the galenic system. The release of the active principle(s) is thus guaranteed after a precontrolled lag time, even if the variation in pH has not intervened as triggering factor, i.e. even if the galenic form has not passed from the stomach to the intestine. These microcapsules of diameter between 200 and 600 microns are characterized by a coating film based on a hydrophilic polymer A of EUDRAGIT®L type combined with a hydrophobic compound B, such as a plant wax (LUBRITAB®) with a melting temperature of between 40 and 90° C., the ratio B/A=0.2-1.5. These microcapsules have an in vitro dissolution behavior such that, at constant pH 1.4, a lag phase of between 1 and 5 hours is observed, followed by an active principle release phase, and such that the change from pH 1.4 to pH 6.8 brings about release of the active principle without any lag time in vitro.

The multimicroparticulate galenic system according to PCT patent application WO-A-03/030878 also makes it possible to adjust the lag time preceding the release of the AP in the stomach by taking into consideration the physiological conditions of the gastrointestinal tract in humans. This advantageous method is thus a means of minimizing the interindividual variability of absorption of the AP. In fact, according to the well-known results of Davis et al., J. of Controlled Release, 2, 27-38 (1985), the residence time, in the stomach, of a preparation is very variable, of the order of from 0.5 to 10 hours. Now, specifically, the abovementioned system makes it possible to release the active principle in the stomach after a given constant lag time within this range of 0.5-10 hours, such that, from one individual to the other, or even from one day to the other for the same individual, the medicament action time is the same.

In fact, the microparticulate oral galenic form for the delayed and controlled release of AP, according to WO-A-03/030878, simultaneously has the following properties:
the release of the AP can be triggered in two ways:
   by “time-dependent” release when the amount of time that the particles spend in the stomach exceeds a period of 5 hours;
   by “pH-dependent” release, which begins without any lag time when the system penetrates the intestine and the pH increases. These two AP-release-triggering factors placed in series guarantee release of the AP after a precontrolled lag time, even if the variation in pH has not intervened as triggering factor;
it consists of a plurality of small microcapsules of coated AP;
the mass fraction of coating excipients is limited.

It should be noted that the problem of maintaining a high plasma profile for as long as possible can be solved, in accordance with the invention according to WO-A-03/030878, by using a mixture of microcapsules having different delayed and controlled release profiles. This makes it possible to produce release profiles which exhibit several waves of release or which ensure, by means of an appropriate control of the various fractions, a constant level of plasma concentration of the active principle(s).

### TECHNICAL PROBLEM

It nevertheless remains that this microparticulate oral galenic form for the delayed and controlled release of active principle(s), according to WO-A-03/030878, can be further improved.

In fact, it is known that, in order to be released, a microencapsulated active principle must first of all be reached by the fluids of the gastrointestinal tract, which, in order to do this, must cross the coating film of the microcapsules. The microencapsulated active principle can then dissolve in these fluids. The active principle solution thus obtained can subsequently diffuse out of the microcapsules through the coating film(s) of said microcapsules. Thus, in order to obtain a lag time of between 0.5 and 7 h, for example of 2-3 h, it is important for the coating film of the microcapsules to have a sufficient thickness (in μm) and/or to represent a sufficient degree of film-coating DF (in % by weight) so that the entry time of a liquid such as water or a gastrointestinal tract fluid into the microcapsule allows delayed release of the active principle. This minimum thickness can, for example, correspond to a DF of 10-40%, for example.

However, when the thickness of the film-coating is sufficient to result in a lag time, it is possible to note that, in the case of low-solubility active principles, the “time-dependent” release, just like the “pH-dependent” release, of the active principle is still as effective, but becomes slower, which can be detrimental to the bioavailability. For example, at least 80% by weight of the active principle is not released after, for example, 16 h at pH=7.0 in an in vitro dissolution test carried out according to the indications of the European Pharmacopeia 4th edition, entitled: “Dissolution test for solid oral forms”: type II dissolutest carried out under SINK conditions, maintained at 37° C. and stirred at 100 rpm.

There exists, therefore, to date a need for a pharmaceutical composition or a multimicrocapsule oral medicament, for the modified release of active principle(s), which is of the type of those disclosed in WO-A-03/030878 and which improves the latter, making it possible in particular to obtain, for low-solubility active principles, release of the active principle according to a dual “time-dependent” and “pH-dependent” trigger mechanism, with more rapid release times which make it possible to optimize the bioabsorption of the active principle(s), regardless of the mechanism for triggering this release.

### OBJECTIVES

One of the essential objectives of the invention is to provide an oral medicament that is improved in relation to that described in WO-A-03/030878, specifically a multimicrocapsule oral medicament for the modified release of active principle(s), in particular of low-solubility active principle(s), which guarantees correct functioning of the dual “time-dependent” and “pH-dependent” trigger mechanism for the release of the active principle, in particular for low-solubility active principles.

Another essential objective of the invention is to provide a multimicrocapsule oral medicament for the modified release of active principle(s) that makes it possible to judiciously adjust the release kinetics of the active principle all along its window of absorption in the gastrointestinal tract so that the plasma concentration profile is maintained above the effective therapeutic concentration for as long as possible, and in particular for a period of time greater than that of the immediate-release form.

Another essential objective of the invention is to provide a multimicrocapsule oral medicament for the modified release of active principle(s), which provides a suitable solution to the problem of chronotherapy and to the difficulties of compliance relating thereto.

Another essential objective of the invention is to provide a multimicrocapsule oral medicament for the modified release of active principle(s), which makes it possible to readily combine at least two active principles in the same pharmaceutical form.

Another essential objective of the invention is to provide a multimicrocapsule oral medicament for the modified release of active principle(s), which, contrary to the compact monolithic forms, offers a reduced interindividual variability.

Another essential objective of the invention is to provide a multimicrocapsule oral medicament for the modified release of active principle(s), which allows an increase in the gastrointestinal transit time and absorption of the active principle in the upper parts of the gastrointestinal tract.

Another essential objective of the invention is to provide a multimicrocapsule oral medicament for the modified release of active principle(s), it being possible for this medicament to exist in a presentation form that can be administered once a day, which would represent significant progress, in particular in terms of patient compliance.

Another essential objective of the invention is to provide a multimicrocapsule oral medicament for the modified release of active principle(s), which can be produced according to a sound industrial process.

Another essential objective of the invention is to provide a multimicrocapsule oral medicament for the modified release of active principle(s), which is easy to prepare, for example by deposition of a coating by spraying onto microparticles containing low-solubility active principle.

Another essential objective of the invention is to provide a multimicrocapsule oral medicament for the modified release of active principle(s), which is capable of having high contents of active principle(s), for example up to 60% by weight of the microcapsules.

Another essential objective of the invention is to provide an oral medication for the modified release of active principle(s), which contains a plurality of microcapsules and has a dose-independent in vitro active principle release profile.

Another essential objective of the invention is to provide a multimicrocapsule oral medicament for the modified release of active principle(s), in which the microcapsules are nonenteric, i.e. do not release the active principle only when the pH goes from 1.4 to 7.0 (gastric pH= [symbol] intestinal pH).

Another essential objective of the invention is to provide a multimicrocapsule oral medicament for the modified release of active principle(s), which makes it possible to obtain a plasma concentration (C24h) of active principle(s) 24 h after oral administration which is as high as possible.

Another essential objective of the invention is to provide microcapsules for the modified release of active principles, which can be used in particular for preparing a medicament as defined by the specifications stated in the above objectives.

### BRIEF DISCLOSURE OF THE INVENTION

These objectives, among others, are achieved by means of the invention which relates, firstly, to an oral medicament comprising a plurality of microcapsules for the modified release of active principle(s), at least some of said microcapsules consisting individually of a microparticle comprising at least one active principle, in particular at least one low-solubility active principle (with the exclusion of carvedilol), coated with at least one coating for the modified release of the active principle(s), said release being controlled by two distinct trigger mechanisms, one being based on a variation in pH and the other allowing the release of the active principle(s) after a predetermined period of time spent in the stomach,

said coating also conferring on the microcapsules an in vitro dissolution behavior such that:
at constant pH 1.4, the dissolution profile comprises a lag phase less than or equal to 7 hours, preferably less than or equal to 5 hours, and even more preferably between 1 and 5 hours, in duration;
the passing from pH 1.4 to pH 7.0 results in a release phase which begins without any lag time;
   this medicament being characterized
   in that at least some of said microcapsules comprise at least one swelling agent,
   and in that the fraction by weight of the active principle(s) released during the lag phase is less than or equal to 15% by weight per hour, preferably less than or equal to 10% by weight per hour, and even more preferably less than or equal to 5% by weight per hour.

The in vitro dissolution behavior is determined according to the indications of the European Pharmacopeia 4th edition, entitled: “Dissolution test for solid oral forms”: type II dissolutest carried out under SINK conditions, maintained at 37° C. and stirred at 100 rpm.

The medicament according to the invention overcomes the abovementioned technical problem, i.e. the release of low-solubility active principle(s) AP(s) according to a dual “time-dependent” and “pH-dependent” trigger mechanism, thereby accelerating the release of the active principle, in particular compared with the release times obtained by the invention according to WO-A-03/030878. In doing so, the medicament according to the invention ultimately improves the prophylactic and therapeutic efficacy of such low-solubility active principles.

However, the medicament according to the invention is also advantageous in that it offers in particular the following advantages:

possibility of simple combined use of at least two active principle(s);

reduced interindividual variability;

increase in gastrointestinal transit time and absorption of the active principle in the upper parts of the gastrointestinal tract;

proportionality between the dose and the pharmacokinetic profile;

ease of ingestion by the patient and possibility of administration, for example once a day, which ensures that compliance is observed and therefore guarantees efficacy;

reproducibility of the release kinetics, from one industrial batch to the other; industrial development is therefore possible, without this harming the therapeutic performance levels of the encapsulated active principle(s) (for example, low-solubility active principles);

easy and economical preparation, for example by deposition of a coating by spraying onto microparticles containing low-solubility active principle;

possibility of having high contents of active principle(s), for example up to 60% by weight of the microcapsules;

plurality of microcapsules and having a dose-independent in vitro active principle release profile;

nonenteric microcapsules, i.e. which do not release the active principle only when the pH goes from 1.4 to 7.0 (gastric pH= [symbol] intestinal pH);

plasma concentration of active principle(s) 24 h after oral administration close to or greater than that which would be obtained with an immediate-release form taken in several doses.

### DETAILED DISCLOSURE OF THE INVENTION

In accordance with the invention, the swelling agent comprises at least one hydrophilic pharmaceutically acceptable compound which exhibits a degree of swelling in water at 25° C. of greater than or equal to 10% by weight, preferably greater than or equal to 15% by weight, and even more preferably greater than or equal to 20%.

According to a notable characteristic of the invention, the swelling agent is chosen from those which allow the microcapsules to release at least 50% by weight of the active principle, after 16 h at pH=1.4, and after a lag phase of less than or equal to 7 hours, preferably less than or equal to 5 hours, and even more preferably between 1 and 5 hours in duration, in an in vitro dissolution test carried out according to the indications of the European Pharmacopeia 4th edition, entitled: “Dissolution test for solid oral forms”: type II dissolutest carried out under SINK conditions, maintained at 37° C. and stirred at 100 rpm.

In accordance with the invention, it is possible to adjust the rate of release at pH=1.4 of the active principle(s) from the microcapsules by judiciously selecting the concentration (Cd) of swelling agent.

When the swelling agent is in microparticulate form, the size (Td) of the particles of swelling agent is advantageously selected within the average diameter ranges, in μm, of between 5 and 200 μm, and preferably between 10 and 50 μm.

The concentration (Cd) of swelling agent is selected within the following ranges of % by weight relative to the total mass of the microcapsules:

3≦Cd≦40,

preferably, 4≦Cd≦30,

and even more preferably, 5≦Cd≦25.

According to a preferred embodiment of the invention, the swelling agent is chosen from the group of following products:
crosslinked polyvinylpyrrolidones (e.g. polyplasdone or crospovidone),
crosslinked carboxyalkylcelluloses: crosslinked carboxymethylcelluloses (e.g. crosslinked sodium croscarmellose),
and also high molar mass hydrophilic polymers (greater than or equal to 100 000 D) such as:
   polyvinylpyrrolidones,
   polyalkylene oxides (e.g. polyethylene oxide or polypropylene oxide),
   (hydroxy)(alkyl)celluloses (e.g. hydroxypropylcellulose, hydroxypropylmethylcellulose),
   carboxyalkylcelluloses (e.g. carboxymethylcellulose),
   celluloses (powder or microcrystalline),
   modified starches (for example modified with sodium glycolate),
   natural starches (for example from maize, wheat or potato),
   sodium alginate,
   potassium polacriline,
   and mixtures thereof.

Even more preferably, the swelling agent is chosen from the subgroup of following products:
crosslinked polyvinylpyrrolidones (e.g. polyplasdone or crospovidone), and
crosslinked carboxyalkylcelluloses: crosslinked carboxymethylcelluloses (e.g. crosslinked sodium croscarmellose).

In order to overcome the eventuality in which active principles (for example, low-solubility active principles) would be poorly wetted by water and would therefore have a tendency to agglomerate, it is proposed, according to an advantageous variant of the invention, to make sure that the medicament comprises at least one wetting agent, preferably chosen from the group of following products:
anionic surfactants, preferably in the subgroup of alkali metal or alkaline earth metal salts of fatty acids, stearic acid and/or oleic acid being preferred,
and/or nonionic surfactants, preferably in the following subgroup:
   polyoxyethylenated oils, preferably polyoxyethylenated hydrogenated castor oil,
   polyoxyethylene-polyoxypropylene copolymers,
   polyoxyethylenated sorbitan esters,
   polyoxyethylenated castor oil derivatives,
   stearates, preferably calcium stearate, magnesium stearate, aluminum stearate or zinc stearate,
   stearyl fumarates, preferably sodium stearyl fumarate,
   glyceryl behenate,
   and mixtures thereof.

Advantageously, the medicament according to the invention comprises microcapsules of active principle(s), capable of releasing at least 80% by weight of the active principle(s), after 12 h at pH=7.0, in an in vitro dissolution test carried out according to the indications of the European Pharmacopeia 4th edition, entitled: “Dissolution test for solid oral forms”: type II dissolutest carried out under SINK conditions, maintained at 37° C. and stirred at 100 rpm.

The medicament according to the invention is multimicrocapsular, i.e. it comprises, inter alia, microcapsules consisting of microparticles of coated or film-coated active principle. These microparticles of active principle may be, for example, crude (pure) active principle in pulverulent form, matrix granules of active principle with various other ingredients, or alternatively neutral microspheres, for example made of cellulose or of sugar, coated with at least one layer comprising active principle.

The modified-release microcapsules of active principle can be compared to microunits containing at least one active principle and forming at least some of the constitutive elements of the medicament according to the invention.

Each microcapsule can contain one or more active principles that are identical to or different than one another.

The medicament according to the invention can comprise microunits of active principle other than microcapsules. They could, for example, be microcapsules for the immediate release of active principle(s). The latter may, for example, be uncoated microparticles of active principle(s) of the same type as those that can be used in the preparation of the microcapsules according to the invention.

Each microparticle can comprise one or more active principles that are identical to or different than one another.

In addition, the collection of microunits (microparticles and/or microcapsules) constituting the medicament according to the invention can be made up of various populations of microunits, these populations differing from one another at least by virtue of the nature of the active principle(s) contained in these microunits and/or by virtue of the composition of the coating.

As regards the structure of the microcapsules used in the medicament according to the invention, two preferred embodiments of the structure of the microcapsules are described in detail hereinafter, in a nonlimiting capacity.

According to a first embodiment, at least some of the microcapsules for the modified release of active principle(s) each comprise:
a microparticle of active principle(s), coated with
at least one coating for the modified release of the active principle(s).

Preferably, the microparticle of active principle(s) is a granule comprising the active principle(s) and one or more pharmaceutically acceptable excipients.

Advantageously, the swelling agent(s) is (are) contained in the microparticle (e.g. granule).

As regards the wetting agent(s), it (they) is (are) preferably contained in the microparticle (e.g. granule) and/or in the coating for the modified release of the active principle(s).

According to a second embodiment, at least some of the microcapsules for the modified release of active principle(s) each comprise:
a neutral core,
at least one active layer comprising the active principle(s) and coating the neutral core, and
at least one coating for the modified release of the active principle(s).

According to a first possibility, the neutral core contains sucrose and/or dextrose and/or lactose.

According to a second possibility, the neutral core is a cellulose microsphere.

Advantageously, the neutral core has an average diameter of between 1 and 800 μm, and preferably between 20 and 500 μm.

The active layer can optionally comprise, in addition to the active principle(s), one or more pharmaceutically acceptable excipients.

Advantageously, the swelling agent(s) is (are) contained in the active layer.

For example, this active layer comprises active principle, at least one swelling agent, at least one binder and at least one surfactant.

As regards the wetting agent(s), it (they) is (are) preferably contained in the active layer.

With regard now to the composition of the coating of the microcapsules for the modified release of active principle(s), the present invention also consists in selecting microcapsules having the following specificities:
the coating for the modified release of the active principle(s) comprises a composite material
   comprising:
      at least one hydrophilic polymer A bearing groups that are ionized at neutral pH,
      at least one hydrophobic compound B,
   representing a mass fraction (% weight relative to the total mass of the microcapsules)≦40; and
their average diameter is less than 2000 μm, and preferably between 50 and 800 μm, and even more preferably between 100 and 600 μm.

According to another advantageous characteristic, the composite material AB of the coating for the modified release of the low-solubility active principle is such that:
the weight ratio B/A is between 0.2 and 1.5, preferably between 0.5 and 1.0,
and the hydrophobic compound B is selected from products that are crystalline in the solid state and have a melting point MpB≧40° C., preferably MpB≧50° C., and even more preferably 40° C.≦MpB≦90° C.

According to a favored embodiment, the hydrophilic polymer A is chosen from:
A.a copolymers of (meth)acrylic acid and of a (meth)acrylic acid alkyl ester, and mixtures thereof;
A.b cellulose derivatives, preferably cellulose acetates, cellulose phthalates, cellulose succinates and mixtures thereof, and even more preferably hydroxypropylmethylcellulose phthalates, hydroxypropylmethylcellulose acetates, hydroxypropylmethylcellulose succinates and mixtures thereof;
and mixtures thereof.

The polymers A that are even more preferred are copolymers of (meth)acrylic acid and of (meth)acrylic acid alkyl (e.g. C₁-C₆ alkyl) esters. These copolymers are, for example, of the type such as those sold by the company Röhm Pharma Polymers under the registered trademarks EUDPAGIT®, of the series L and S (such as, for example, EUDRAGIT® L100, S100, L30 D-55 and L100-55). These copolymers are anionic enteric copolymers that are soluble in an aqueous medium at pHs above those encountered in the stomach.

Still according to the favored embodiment, the compound B is chosen from the group of following products:
B.a plant waxes taken on their own or as mixtures with one another;
B.b hydrogenated plant oils taken on their own or as mixtures with one another;
B.c mono- and/or di- and/or triesters of glycerol and of at least one fatty acid;
B.d mixtures of monoesters, of diesters and of triesters of glycerol and of at least one fatty acid;
B.e and mixtures thereof.

Even more preferably, the compound B is chosen from the group of following products: hydrogenated cottonseed oil, hydrogenated soya bean oil, hydrogenated palm oil, glyceryl behenate, hydrogenated castor oil, tristearin, tripalmitin, trimyristin, yellow wax, hard fat or fat that can be used as bases for suppositories, anhydrous dairy fats, lanolin, glyceryl palmitostearate, glyceryl stearate, lauryl macrogolglycerides, cetyl alcohol, polyglyceryl diisostearate, diethylene glycol monostearate, ethylene glycol monostearate, Omega 3 and any mixture thereof,

preferably from the subgroup of following products: hydrogenated cottonseed oil, hydrogenated soya bean oil, hydrogenated palm oil, glyceryl behenate, hydrogenated castor oil, tristearin, tripalmitin, trimyristin and any mixture thereof.

In practice, and without it being limiting, compound B is preferably chosen:
from the group of products sold under the following trademarks: Dynasan®, Cutina®, Hydrobase®, Dub®, Castorwax®, Croduret®, Compritol®, Sterotex®, Lubritab®, Apifil®, Akofine®, Softtisan®, Hydrocote®, Livopol®, Super Hartolan®, MGLA®, Corona®, Protalan®, Akosoft®, Akosol®, Cremao®, Massupol®, Novata®, Suppocire®, Wecobee®, Witepsol®, Lanolin®, Incromega®, Estaram®, Suppoweiss®, Gelucire®, Precirol®, Emulcire®, Plurol diisostearique®, Geleol®, Hydrine® and Monthyle® and mixtures thereof;
and also from the group of additives for which the codes are the following: E 901, E 907, E 903 and mixtures thereof;
and, preferably from the group of commercial products sold under the following trademarks: Dynasan® P60, Dynasan® 114, Dynasan® 116, Dynasan® 118, Cutina® HR, Hydrobase® 66-68, Dub® HPH, Compritol® 888, Sterotex® NF, Sterotex® K, Lubritab® and mixtures thereof.

According to another advantageous characteristic of the invention, the coating for the modified release of the low-solubility active principle is free of talc.

According to another notable characteristic that is the result of the preparation of the microcapsules, the active principle is deposited onto the core by techniques known to those skilled in the art, for example the fluidized air bed spray coating technique, wet granulation, compacting, extrusion-spheronization, etc.

Advantageously, the coating of the microcapsules can comprise, in addition to the essential constituents A and B, other conventional ingredients known to those skilled in the art, such as, in particular:
dyes,
plasticizers, for instance dibutyl sebacate,
hydrophilic compounds, for instance cellulose and derivatives thereof or polyvinylpyrrolidone and derivatives thereof,
and mixtures thereof.

Without it being limiting and according to an even more preferred embodiment, the coating of the microcapsules for the modified release of active principle comprises a single composite AB coating film.

In quantitative terms, the monolayer of coating can represent, for example, at most 40%, preferably at most 30% by weight of the microcapsules. Such a limited degree of coating makes it possible to prepare galenic units that each contain a high dose of active principle, without exceeding a size that would be completely unacceptable with regard to swallowing. The compliance with and therefore the success of the treatment can only be improved by this.

The mechanism for triggering the release of the low-solubility active principle without any variation in pH, after a predetermined residence time in the stomach, results in particular from the control of the rate of hydration of the microcapsules and/or of the dissolution of one or more components of the microcapsules. For example, and without wishing to be limiting, the hydration of the microcapsule can be controlled:
by the presence, in the microcapsules, of hydrophilic products which make it possible to adjust the osmotic pressure or to cause swelling of the microcapsules;
or by regulating the water-permeability of the coating film;
or by creating a microporosity in the coating film;
or even by hydration or dissolution of a compound of the coating film.

One of the determining advantages of the multimicrocapsule galenic system for the delayed and controlled release of active principle(s), for example of low-solubility principle(s), according to the invention, is to involve, in vivo, two factors that trigger the release of the active principle(s), for example of the low-solubility principle(s), in the gastrointestinal tract, i.e.:
the period of time spent in the stomach: “time-triggered” release,
the variation in pH: “pH-triggered” release.

These two factors that trigger the release of active principle(s), for example of low-solubility principle(s), are in series such that they confer very safe use on the galenic system. The release of the active principle(s), for example of the low-solubility principle(s), is thus guaranteed after a precontrolled lag time, even if the variation in pH has not intervened as triggering factor. The problems of interindividual variability are thus overcome. The therapeutic efficacy of the medicament comprising such a galenic system is ensured, observing a predetermined chronobiology adapted to the targeted therapeutic performance level.

In addition, for any active principle (e.g. low-solubility active principle) the window of absorption of which is limited to the upper parts of the gastrointestinal tract, it is particularly advantageous for the form for the delayed and then prolonged and controlled release to be made up of a plurality of microcapsules. In fact, for such a galenic form, the dose of active principle (e.g. low-solubility active principle) to be administered is divided up between a large number of microcapsules (typically 5000-50 000) and as a result provides the following intrinsic advantages:
The period of time spent by the microcapsules in the upper parts of the gastrointestinal tract can be prolonged, thereby ensuring an increase in the amount of time spent by the low-solubility active principle in passing before the windows of absorption and thus maximizing the bioavailability of the low-solubility active principle.
The use of a mixture of microcapsules with different delayed- and controlled-release profiles makes it possible to produce release profiles which have several waves of release or which ensure, through an appropriate control of the various fractions, a constant level of plasma concentration of the low-solubility active principle.
There is less variability in the gastric emptying since the emptying which takes place here over a large number of particles is statistically more reproducible.
Contact between the tissues and a high dose of low-solubility active principle, “dose dumping”, is prevented. Each microcapsule in fact contains only a very low dose of low-solubility active principle. One is thus free of the risk of tissue deterioration due to local overconcentration of aggressive low-solubility active principle.
It is possible to provide these microcapsules in the form of a sachet, gelatin capsule or tablet. When the dose of low-solubility active principle is high (500 mg or more), monolithic forms are too large in size to be readily swallowed. It is then particularly advantageous to have a microparticulate form which ensures the delayed and controlled release of the active principle (in particular low-solubility active principle) and which those skilled in the art can produce in the form of disintegratable tablets or of sachets.

The multimicrocapsule galenic system according to the invention makes it possible to ensure, in a reliable manner, a delayed and controlled release of the low-solubility active principle in the gastrointestinal tract by virtue of two triggers, and to thus escape the inter- and intraindividual variability of the conditions of gastric emptying, while at the same time being economically viable and easy to ingest (optimized compliance).

Another subject of the invention relates to an oral medicament comprising a plurality of microcapsules for the modified release of active principle(s), at least some of said microcapsules consisting individually of a microparticle comprising at least one active principle, in particular at least one low-solubility active principle (with the exclusion of carvedilol), coated with at least one coating for the modified release of the active principle(s), said release being controlled by two distinct trigger mechanisms, one based on a variation in pH and the other allowing the release of the active principle(s) after a predetermined period of time spent in the stomach,

said coating:
also conferring on the microcapsules an in vitro dissolution behavior (produced according to the indications of the European Pharmacopeia 4th edition, entitled: “Dissolution test for solid oral forms”: type II dissolutest carried out under SINK conditions, maintained at 37° C. and stirred at 100 rpm), such that:
   at constant pH 1.4, the dissolution profile comprises a lag phase less than or equal to 7 hours, preferably less than or equal to 5 hours, and even more preferably between 1 and 5 hours, in duration;
   at least 50% by weight of the active principle(s) is released after 16 h at pH 1.4;
   the passing from pH 1.4 to pH 7.0 results in a release phase which begins without any lag time;
and comprising a composite material comprising at least one hydrophilic polymer A bearing groups that are ionized at neutral pH and at least one hydrophobic compound B;
   this medicament being characterized
in that at least some of said microcapsules comprise at least one release helper capable of increasing the permeability of the coating for the modified release of the active principle(s),
and in that the fraction by weight of the active principle(s) released during the lag phase is less than or equal to 15% by weight per hour, preferably less than or equal to 10% by weight per hour, and even more preferably less than or equal to 5% by weight per hour.

It has in fact appeared to be useful in accordance with the invention to provide for, in the microcapsules, one or more helpers suitable for increasing the permeability of the coating, so as to reduce the release time, in particular for low-solubility active principles.

Advantageously, the release helper consists of at least one swelling agent, as defined above.

The medicament according to this other subject of the invention is also remarkable in that the coating of the microcapsules included in this medicament confers on said microcapsules an in vitro dissolution behavior (produced according to the indications of the European Pharmacopeia 4th edition, entitled: “Dissolution test for solid oral forms”: type II dissolutest carried out under SINK conditions, maintained at 37° C. and stirred at 100 rpm),

such that at least 50% by weight of the active principle(s) is released after 16 h at pH 1.4.

The fact that the medicament according to the invention consists of a plurality of microunits makes it possible to obtain another essential characteristic of the invention, according to which the medicament comprises a mixture of various populations of microunits containing active principle(s) (with the exclusion of carvedilol), these populations differing from one another by virtue of their respective in vitro dissolution profiles, for at least one pH value of between 1.4 and 7.4.

This essential characteristic makes it possible to obtain an increase in the bioabsorption time of the active principle(s) and therefore in the time during which the plasma concentration is greater than the minimum effective concentration of this active principle.

In fact, the mixture of various populations of microunits (e.g. microcapsules for the modified release of active principle and, optionally, microparticles for the immediate release of active principle) brings about a preferential release of the active principle(s) at different sites of the gastrointestinal tract, over the entire extent of the window of bioabsorption of the active principle(s) in the gastrointestinal tract.

According to an advantageous embodiment of this characteristic of a mixture of various populations of microunits, the medicament according to the invention is characterized in that the microunits are microcapsules for the modified release of active principle(s) and, optionally, microunits for the immediate release of active principle(s).

Advantageously, the populations of microcapsules for the modified release of active principle(s) differ by virtue of their respective trigger pHs.

The populations of microcapsules for the modified release of active principle(s) can differ not only by virtue of their respective trigger pHs, but also by virtue of their respective trigger times, or even by virtue of their respective trigger pHs and times.

According to a preferred embodiment of this characteristic of a mixture of populations, the medicament comprises:
i. at least one population of microunits containing immediate-release active principle;
ii. at least one population P1 of microcapsules for the modified release of active principle(s); and
iii. at least one population P2 of microcapsules for the modified release of active principle(s);
   and, moreover, the respective trigger pHs of P1 and of P2 differ by at least 0.5 pH unit, preferably by at least 0.8 pH unit, and even more preferably by at least 0.9 pH unit.

According to a preferred arrangement relating to the trigger pHs that differentiate the various populations of microcapsules for the modified release of active principle(s), said respective trigger pHs are between 5 and 7.

According to another variant of the preferred embodiment of this characteristic of a mixture of populations, the medicament comprises:
i. at least one population of microunits containing immediate-release active principle(s);
ii. at least one population P1′ of microunits containing active principle(s) made up of microcapsules for the modified release of the active principle(s), the trigger pH of which is equal to 5.5; and
iii. at least one population P2′ of microunits containing active principle(s) made up of microcapsules for the modified release of the active principle(s), the trigger pH of which is equal to 6.0 or 6.5.

The release profile (measured in an in vitro release test as defined above) of the modified-release microcapsules used in the abovementioned mixtures of various populations of microunits (for example, P1 and P2 or P1′ and P2′) may, for example, be as indicated hereinafter:

less than 20% of the active principle(s) is released after 2 hours at pH=1.4;

at least 50% of the active principle(s) is released after 16 hours at pH=1.4.

When the medicament according to the invention comprises at least one population of microunits containing immediate-release active principle(s), this population can advantageously be defined by its behavior in an in vitro dissolution test, said behavior being such that at least 80% of the active principle(s) is released in 1 hour at any pH between 1.4 and 7.4.

According to an advantageous characteristic of the invention, the proportion of low-solubility active principle(s) in the microunits containing active principle(s) (expressed as % by weight on a dry weight basis, relative to the total mass of the microunits) is between 5 and 80, preferably between 10 and 70, and even more preferably between 15 and 60.

Preferably, the microunits containing immediate-release active principle(s) are uncoated microparticles.

Without wishing to be limiting, it should nevertheless be underlined that the medicament according to the invention is particularly advantageous in that it can be in the form of a single daily oral dose comprising:
from 5000 to 500 000 microunits containing active principle(s), or
from 5000 to 500 000 microcapsules for the modified release of active principle(s).

This plurality of microcapsules, illustrated by the numeric examples mentioned above, constitutes a galenic form that is entirely well tolerated by the mammalian organism.

These microcapsules are all the more advantageous since they are manufactured simply and economically according to techniques well known to those skilled in the art, for example the fluidized air bed spray coating technique, wet granulation, compacting, extrusion-spheronization, etc.

For further details on the preparation of these microcapsules, in particular in their preparation form with a neutral core coated with at least one active layer comprising active principle(s) and with at least one outer coating for the modified release of the active principle(s), reference will be made to the content of PCT application WO-A-FR03/030878, the content of which is integrated into the present disclosure by way of reference.

The medicament in multimicroparticulate oral galenic forms according to the invention can be a tablet, advantageously an orodispersible tablet, a powder, a liquid suspension or a gelatin capsule containing microcapsules.

In other words, this medicament can be in the form of a sachet of microcapsule powder, of a liquid suspension of microcapsules, of a tablet obtained from microcapsules or a gelatin capsule containing microcapsules.

These tablets, these gelatin capsules, these powders and these suspensions can consist of mixtures of the various populations of microunits, and in particular of microcapsules of active principle(s) according to the invention, preferably combining therewith microunits or microparticles for the immediate release of low-solubility active principle (for example granules).

Moreover, the invention is directed toward the use of the microcapsules for the modified release of active principle(s) as defined above and, optionally, of microunits containing immediate-release active principle(s), for the preparation of pharmaceutical or dietetic, microparticulate oral galenic forms, preferably in the form of advantageously orodispersible tablets, of powders or of gelatin capsules.

Furthermore, the invention relates to the microcapsules as defined above, taken as such.

Preferably, the active principle(s) can be chosen from at least one of the following major varieties of active substances, e.g.: antiulcer agents, antidiabetic agents, anticoagulants, antithrombotics, blood-lipid-lowering agents, antiarrhythmics, vasodilators, anti-angina agents, antihypertensives, vasoprotectors, fertility promoters, inhibitors and inducers of uterine labor, contraceptives, antibiotics, antifungals, antivirals, anticancer agents, anti-inflammatories, analgesics, antiepileptics, antiparkinsonian agents, neuroleptics, hypnotics, anxiolytics, psychostimulants, antimigraine agents, antidepressants, antitussives, antihistamines or anti-allergic agents, agents for combating congestive heart failure, angina pectoris, left ventricular hypertrophy, cardiac arrhythmias, myocardial infarction, reflex tachycardia, ischemic heart disease, atheromatosis, diabetes mellitus-related hypertension, portal hypertension, vertigo, abradycardia, arterial hypotension, water and sodium retention, acute renal insufficiency, orthostatic hypotension and cerebral congestion, and mixtures thereof.

As examples of active principles that may be contained in the medicament according to the invention, mention may be made of those chosen from the group of following compounds: acetylsalicylic acid, carbamazepine pentoxifylline, prazosine, acyclovir, nifedipine, diltiazem, naproxen, ibuprofen, flurbiprofen, ketoprofen, fenoprofen, indomethacin, diclofenac, fentiazac, estradiol valerate, metoprolol, sulpiride, captopril, cimetidine, zidovudine, nicardipine, terfenadine, atenolol, salbutamol, carbamazepine, ranitidine, enalapril, simvastatin, fluoxetine, alprazolam, famotidine, ganciclovir, famciclovir, spironolactone, 5-asa, quinidine, perindopril, morphine, pentazocine, paracetamol, omeprazole, lansoprazole, metoclopramide, aminosalicylic acid, nalidixic acid, amoxicillin, amoxicillin and potassium clavulanate, ampicillin, ampicillin and sulbactam, azithromycin, bacampicillin, carbenicillin-indanyl-sodium (and other carbenicillin salts), capreomycin, cefadroxil, cefazoline, cephalexine, cephalothine, cephapirine, cephacelor, cephprozile, cephadrine, cefamandole, cefonicide, ceforanide, cefuroxime, cefixime, cefoperazone, cefotaxime, cefpodoxime, ceftaxidime, ceftibuten, ceftizoxime, ceftriaxone, cefepime, cefmetazole, cefotetan, cefoxitin, ciprofloxacin, clarithromycin, clindamycin, clofazimine, cloxacillin, cotriamoxazole, cycloserine, dicloxacillin, dirithromycin, erythromycin (and erythromycin salts such as estolate, ethyl succinate, gluceptate, lactobionate, stearate), ethambutol-HCl and other salts, ethionamide, fosfomycin, imipenem, isoniazide, levofloxacin, lomefloxacin, loracarbef, methicillin, methenamine, metronidazole, metoclopramide, mezlocillin, nafcillin, nitrofurantoin, norfloxacine, novobiocine, ofloxacine, oxacillin, penicillin V, penicillin salts, penicillin complexes, pentamidine, piperacillin, piperacillin and tazobactam, sparfloxacine, sulfacytine, sulfamerazine, sulfamethazine, sulfamethixole, sulfasalazine, sulfisoxazole, sulfapyrizine, sulfadiazine, sulfinethoxazole, sulfapyridine, ticarcillin, ticarcillin and potassium clavulanate, trimethoprim, trimetrexate, troleanomycin, vancomycin, verapamil and mixtures thereof.

According to a specific but nonlimiting variant of the invention, the active principle(s) is (are) one of the low-solubility active principle(s), for example chosen from the active principles as mentioned above (taken by themselves or as a mixture with one another).

The active principles to which the present invention also relates may also be nutritional and/or dietetic supplements or mixtures thereof, for instance vitamins, amino acids, antioxidants or trace elements, or mixtures thereof.

Finally, the invention is also directed toward a method of therapeutic treatment, characterized in that it consists of an oral administration, according to a given dosage, of the medicament according to the invention as defined above.

The invention will be explained more thoroughly by means of the examples hereinafter, given solely by way of illustration in order to fully understand the invention and to reveal its preparation and/or implementation variants.

### EXAMPLES

### Description of the Figures

FIG. 1: in vitro release profiles of the microcapsules prepared according to comparative example 1.

FIG. 2: in vitro release profiles of the microcapsules prepared according to comparative example 2.

FIG. 3: in vitro release profiles of the microcapsules prepared according to comparative example 3.

FIG. 4: in vitro release profiles of the microcapsules prepared according to comparative example 4.

FIG. 5: in vitro release profiles of the microcapsules prepared according to example 5 and comparison with the release profiles of the microcapsules prepared according to comparative example 1.

FIG. 6: in vitro release profiles of the microcapsules prepared according to example 6 and comparison with the release profiles of the microcapsules prepared according to comparative example 2.

FIG. 7: in vitro release profiles of the microcapsules prepared according to example 7 and comparison with the release profiles of the microcapsules prepared according to comparative example 3.

FIG. 8: release profiles of the microcapsules prepared according to examples 8, 9 and 10 at pH 1.4.

FIG. 9: release profiles of the microcapsules prepared according to examples 8, 9 and 10, measured for 2 h at pH 1.4 and then at pH 6.8.

FIG. 10: release profiles of the microcapsules prepared according to examples 10, 11, 12 and 13 at pH 1.4.

FIG. 11: release profiles of the microcapsules prepared according to example 14.

FIG. 12: release profiles of the microcapsules prepared according to example 15.

FIG. 13: release profiles of the microcapsules prepared according to example 16.

FIG. 14: release profiles of the microcapsules prepared according to example 17.

The examples below relate to the following active principles:

| Active principle | Solubility (g/l) |
|---|---|
| Spironolactone | 0.02 |
| Lansoprazole | 0.05 |
| Nitrofurantoin | 0.3 |
| Amoxicillin trihydrate | 3.0 |
| Acyclovir | 10.0 |

Comparative example 1 (spironolactone), comparative example 2 (amoxicillin trihydrate), comparative example 3 (nitrofurantoin) and comparative example 4 (carvedilol) illustrate formulations for the delayed and controlled release of the active principle, obtained according to WO-A-03/030878. However, it would be advantageous to conserve the lag phase while at the same time increasing the rate of release after the lag phase, in order to optimize the bioavailability and the efficacy of the active principle. The microcapsules of comparative examples 1 to 4 do not comprise any swelling agent.

Examples 5 (spironolactone), 6 (amoxicillin trihydrate) and 7 (nitrofurantoin) illustrate formulas according to the invention.

Examples 8, 9 and 10 (acyclovir) show the influence of the amount of swelling agent present in the formulas on the release kinetics at pH 1.4.

Examples 11, 12 and 13 (acyclovir) illustrate a nonexhaustive selection of the swelling agents that may be used in the formulas according to the invention.

Example 14 (acyclovir) illustrates the preparation of microcapsules, combining a wet granulation step and a fluidized air bed coating step.

Example 15 (acyclovir) illustrates the preparation of microcapsules, combining an extrusion/spheronization step and a fluidized air bed coating step.

Example 16 (acyclovir) illustrates the preparation of microcapsules, combining a compacting step and a fluidized air bed coating step.

Example 17 (acyclovir) illustrates the preparation of a medicament composed of the mixture of various types of microunits.

### Comparative Example 1

### Preparation of Microcapsules of Spironolactone Containing No Swelling Agent

### Step 1:

432 g of spironolactone and 48 g of low molar mass hydroxypropylcellulose (Klucel® EF/Hercules) are dispersed in 1120 g of purified water. The suspension is sprayed onto 720 g of neutral microspheres (Asahi-Kasei) in a Glatt GPCG1 spray coater.

### Step 2:

43.2 g of hydrogenated cottonseed oil (Penwest) and 64.8 g of poly(methacrylic acid) (ethyl acrylate) Eudragit® L100-55 (Röhm) are dissolved under hot conditions in isopropanol. The solution is sprayed onto 492 g of previously prepared microparticles.

The microcapsules obtained at the end of the second step were tested in a type II dissolutest in accordance with the European Pharmacopoeia 4th edition, at 37° C. and with stirring at 100 rpm, in the following media:
HCl at pH 1.4,
HCl at pH 1.4 for 2 hours and then KH₂PO₄/NaOH buffer medium, at pH 6.8.

The dissolution profiles are presented in FIG. 1. It is noted that:
at pH 1.4, the release of the active principle is slow after the lag period of approximately 2 hours;
when the pH goes from 1.4 to 6.8, the release kinetics accelerate but remain slow (approximately 8 hours are required in order to release 80% of the active principle).

The novel compositions according to the invention make it possible to accelerate the release profiles at pH 1.4 and at pH 6.8, while at the same time conserving the lag phase at pH 1.4.

### Comparative Example 2

### Preparation of Microcapsules of Amoxicillin Trihydrate Containing No Swelling Agent

### Step 1:

1620 g of amoxicillin trihydrate and 180 g of low molar mass hydroxypropylcellulose (Klucel® EF (Hercules)) are dispersed in 4200 g of purified water. The suspension is sprayed onto 200 g of neutral microspheres (Asahi-Kasei) in a Glatt GPCG1 spray coater.

### Step 2:

120 g of hydrogenated cottonseed oil (Penwest) and 180 g of poly(methacrylic acid) (ethyl acrylate) Acrycoat® L100D (NP Pharm) are dissolved under hot conditions in isopropanol. The solution is sprayed onto 700 g of previously prepared microparticles.

The microcapsules obtained at the end of the second step were tested in a type II dissolutest in accordance with the European Pharmacopoeia 4th edition, at 37° C. and with stirring at 100 rpm, in the following media:
HCl at pH 1.4,
HCl at pH 1.4 for 2 hours and then KH₂PO₄/NaOH buffer medium, at pH 6.8.

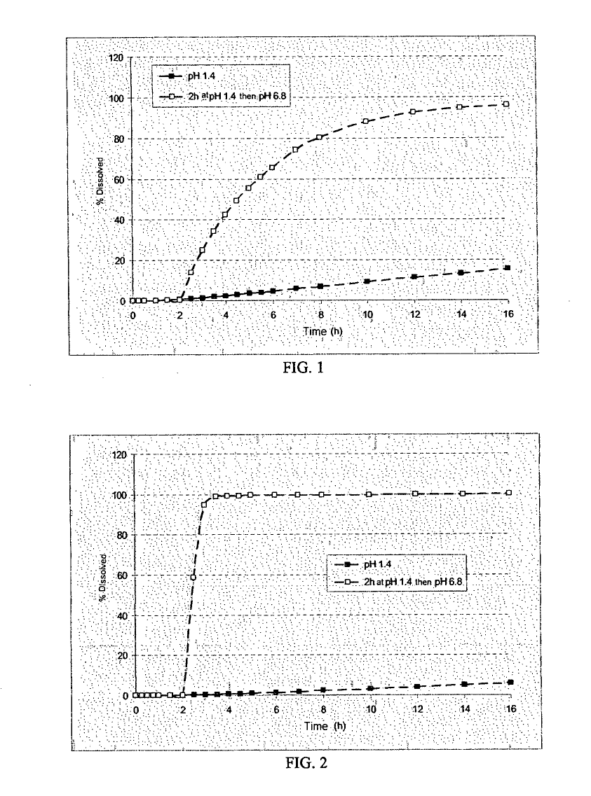

The dissolution profiles are presented in FIG. 2. It is noted that:
at pH 1.4, the release of the active principle is slow after the lag period of approximately 4 hours;
when the pH goes from 1.4 to 6.8, the release kinetics are rapid as expected.

The novel compositions according to the invention make it possible to optimize the release profiles at pH 1.4, while at the same time maintaining a rapid release at pH 6.8 and while at the same time conserving a lag phase at pH 1.4.

### Comparative Example 3

### Preparation of Microcapsules of Nitrofurantoin Containing No Swelling Agent

### Step 1:

640 g of amoxicillin trihydrate and 160 g of low molar mass hydroxypropylcellulose (Klucel® EF/Hercules) are dispersed in 2400 g of purified water. The suspension is sprayed onto 200 g of neutral microspheres (Asahi-Kasei) in a Glatt GPCG1 spray coater.

### Step 2:

40 g of hydrogenated cottonseed oil (Penwest), 5 g of dibutyl sebacate (Morflex) and 55 g of poly(methacrylic acid) (methyl methacrylate) Eudragit® L100 (Röhm) are dissolved under hot conditions in isopropanol. The solution is sprayed onto 900 g of previously prepared microparticles.

The microcapsules obtained at the end of the second step were tested in a type II dissolutest in accordance with the European Pharmacopoeia 4th edition, at 37° C. and with stirring at 100 rpm, in the following media:
HCl at pH 1.4,
HCl at pH 1.4 for 2 hours and then KH₂PO₄/NaOH buffer medium, at pH 6.8.

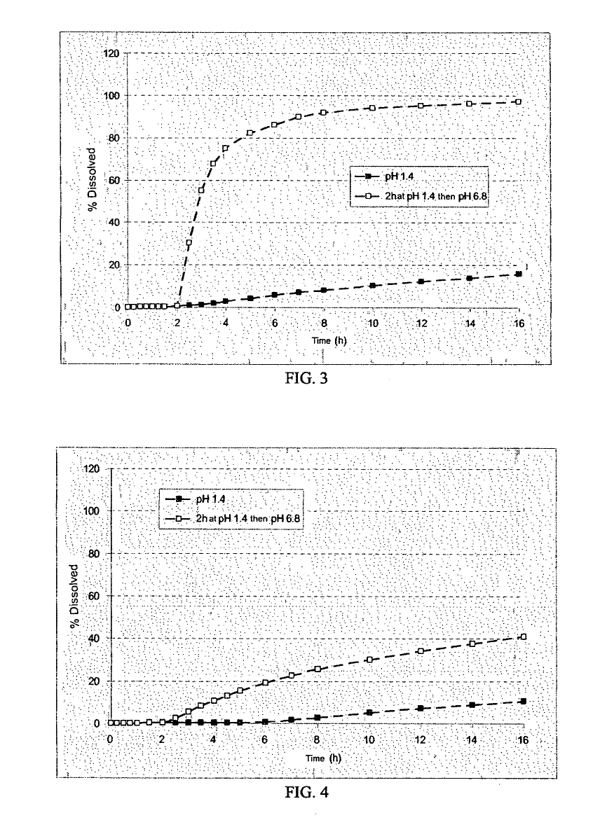

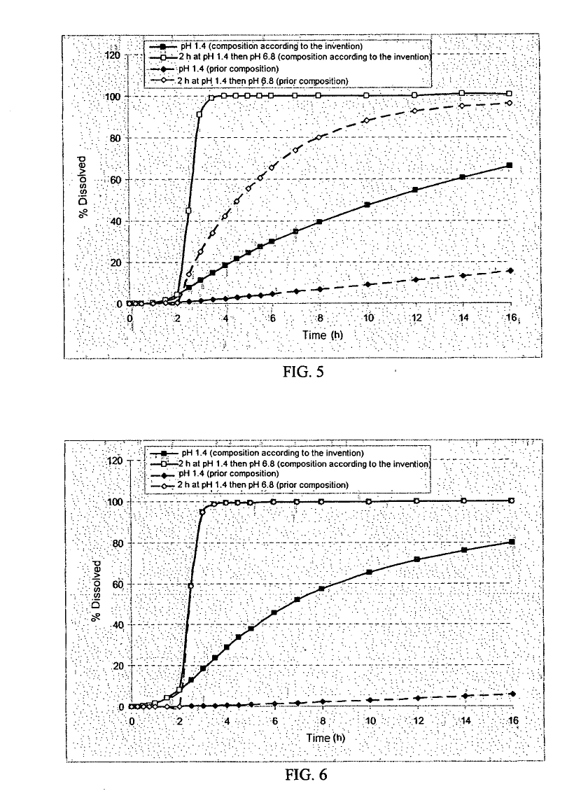

The dissolution profiles are presented in FIG. 3. It is noted that:
at pH 1.4, the release of the active principle is slow after the lag period of approximately 2 hours;
when the pH goes from 1.4 to 6.8, the release kinetics are rapid as expected.

The novel compositions according to the invention make it possible to optimize the release profiles at pH 1.4, while at the same time maintaining a rapid release at pH 6.8 and while at the same time conserving a lag phase at pH 1.4.

### Comparative Example 4

### Preparation of Microcapsules of Carvedilol Phosphate Containing No Swelling Agent

1120 g of carvedilol phosphate and 280 g of Plasdone K29/32® (ISP) are dispersed in 1120 g of purified water. The suspension is sprayed onto 600 g of neutral microspheres (Asahi-Kasei) in a Glatt GPCG1 spray coater.

100 g of hydrogenated cottonseed oil (Penwest) and 150 g of Eudragit® L100-55 (Röhm) are dissolved under hot conditions in isopropanol. The solution is sprayed onto 750 g of previously prepared microparticles.

The microcapsules obtained at the end of the second step were tested in a type II dissolutest in accordance with the Pharmacopoeia, at 37° C. and with stirring at 100 rpm, in the following media:
HCl at pH 1.4,
HCl at pH 1.4 for 2 hours and then KH₂PO₄/NaOH buffer medium, at pH 6.8.

The dissolution profiles are presented in the attached FIG. 4. It is noted that:
at pH 1.4, the release of the active principle is slow after the lag period of approximately 2 hours;
when the pH goes from 1.4 to 6.8, the release kinetics accelerate, but remain slow (at 16 hours, only 40% of the active principle has been released).

### Example 5

### Preparation of Microcapsules of Spironolactone According to the Invention

### Step 1:

216 g of spironolactone, 72 g of low molar mass hydroxypropylcellulose (Klucel® EF/Hercules), 72 g of PEG-40 hydrogenated castor oil (Cremophor RH 40/BASF) and 360 g of crospovidone (Kollidon CL/BASF) are dispersed in 1120 g of purified water. The suspension is sprayed onto 720 g of neutral microspheres (Asahi-Kasei) in a Glatt GPCG1 spray coater.

### Step 2:

43.2 g of hydrogenated cottonseed oil (Penwest) and 64.8 g of poly(methacrylic acid) (ethyl acrylate) Eudragit® L100-55 (Röhm) are dissolved under hot conditions in isopropanol. The solution is sprayed onto 492 g of previously prepared microparticles.

The microcapsules obtained at the end of the second step were tested in a type II dissolutest in accordance with the European Pharmacopoeia 4th edition, at 37° C. and with stirring at 100 rpm, in the following media:
HCl at pH 1.4,
HCl at pH 1.4 for 2 hours and then KH₂PO₄/NaOH buffer medium, at pH 6.8.

The dissolution profiles of example 5 and of comparative example 1 are presented in FIG. 4. It is noted that:
at pH 1.4, approximately 60% of the active principle is released after a lag period of approximately 1.5 hours;
when the pH goes from 1.4 to 6.8, the release kinetics are rapid.

### Example 6

### Preparation of Microcapsules of Amoxicillin Trihydrate According to the Invention

### Step 1:

630 g of amoxicillin trihydrate, 90 g of povidone (plasdone® K29/32 (ISP)) and 180 g of crospovidone (Polyplasdone®/ISP) are dispersed in 2100 g of isopropanol/water mixture (70/30 m/m). The solution is sprayed onto 100 g of neutral microspheres (Asahi-Kasei) in a Glatt® GPCG1 spray coater.

### Step 2:

120 g of hydrogenated cottonseed oil (Abitec) and 160 g of poly(methacrylic acid) (ethyl acrylate) Kollicoat® MAE 100P (BASF) are dissolved under hot conditions in isopropanol. The solution is sprayed onto 700 g of previously prepared microparticles.

The microcapsules obtained at the end of the second step were tested in a type II dissolutest in accordance with the European Pharmacopoeia 4th edition, at 37° C. and with stirring at 100 rpm, in the following media:
HCl at pH 1.4,
HCl at pH 1.4 for 2 hours and then KH₂PO₄/NaOH buffer medium, at pH 6.8.

The dissolution profiles of example 6 and of comparative example 2 are presented in FIG. 6. It is noted that with the composition according to the invention:
the release of the active principle at pH 1.4 was accelerated (guaranteeing triggering of the system after a given period of time and the release of a sufficient amount of active agent, this release taking place over time periods compatible with the absorption times of the active principles in the organism);
when the pH goes from 1.4 to 6.8, rapid release kinetics are maintained.

### Example 7

### Preparation of Microcapsules of Nitrofurantoin According to the Invention

### Step 1:

400 g of nitrofurantoin, 200 g of povidone (plasdone® K29/32/ISP), 50 g of PEG-40 hydrogenated castor oil (BASF) and 350 g of crospovidone (Polyplasdone®/ISP) are suspended in 2500 g of purified water. The solution is sprayed onto 1000 g of neutral microspheres (Asahi-Kasei) in a Glatt® GPCG1 spray coater.

### Step 2:

120 g of hydrogenated cottonseed oil (Abitec) and 160 g of poly(methacrylic acid) (ethyl acrylate) Acrycoat® L100D (NP Pharm) are dissolved under hot conditions in isopropanol. The solution is sprayed onto 700 g of previously prepared microparticles.

The microcapsules obtained at the end of the second step were tested in a type II dissolutest in accordance with the European Pharmacopoeia 4th edition, at 37° C. and with stirring at 100 rpm, in the following media:
HCl at pH 1.4,
HCl at pH 1.4 for 2 hours and then KH₂PO₄/NaOH buffer medium, at pH 6.8.

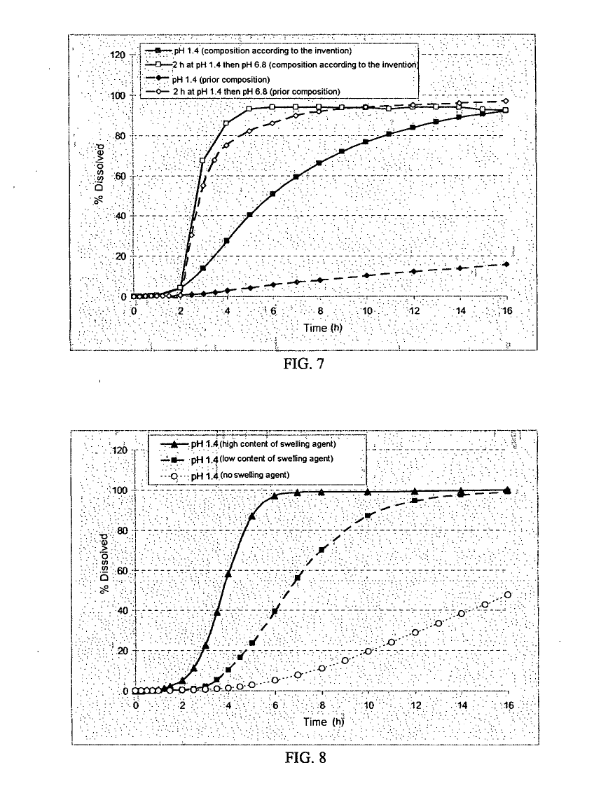

The dissolution profiles of example 7 and of comparative example 3 are presented in FIG. 7. It is noted that, with the composition according to the invention:
the release of the active principle at pH 1.4 was accelerated (guaranteeing triggering of the system after a given period of time and the release of a sufficient amount of active agent, this release taking place over time periods compatible with the absorption times of the active principles in the organism);
when the pH goes from 1.4 to 6.8, rapid release kinetics are maintained.

### Comparative example 8

### Preparation of Microcapsules of Acyclovir Containing No Swelling Agent

### Step 1:

75 g of acyclovir and 75 g of povidone (Plasdone® K29/32/ISP) are dissolved in 833 g of isopropanol. The solution is sprayed onto 850 g of neutral microspheres (NP Pharm) in a Glatt® GPCG3 spray coater.

### Step 2:

93.3 g of hydrogenated soybean oil (Abitec) and 140 g of poly(methacrylic acid) (methyl methacrylate) Eudragit® L100 (Röhm) are dissolved under hot conditions in isopropanol. The solution is sprayed onto 700 g of previously prepared microparticles.

### Example 9

### Preparation of Microcapsules of Acyclovir Containing a Small Amount of Swelling Agent (Crospovidone®)

### Step 1:

375 g of acyclovir, 50 g of low molar mass hydroxypropylcellulose (Klucel® EF (Hercules)) and 75 g of crospovidone (Polyplasdone®/ISP are suspended in 1200 g of purified water. The solution is sprayed onto 500 g of neutral microspheres (NP Pharm) in a Glatt® GPCG3 spray coater.

### Step 2:

100 g of hydrogenated cottonseed oil (Penwest) and 150 g of poly(methacrylic acid) (ethyl acrylate) Eudragit® L100-55 (Röhm) are dissolved under hot conditions in ethanol. The solution is sprayed onto 750 g of previously prepared microparticles.

### Example 10

### Preparation of Microcapsules of Acyclovir Containing a Larger Amount of Swelling Agent (Crospovidone®)

### Step 1:

300 g of acyclovir, 50 g of low molar mass hydroxypropylcellulose (Klucel® EF (Hercules)) and 150 g of crospovidone (Polyplasdone®/ISP are suspended in 1200 g of purified water. The solution is sprayed onto 500 g of neutral microspheres (NP Pharm) in a Glatt® GPCG3 spray coater.

### Step 2:

100 g of hydrogenated cottonseed oil (Penwest) and 150 g of poly(methacrylic acid) (ethyl acrylate) Eudragit® L100-55 (Röhm) are dissolved under hot conditions in ethanol. The solution is sprayed onto 750 g of previously prepared microparticles.

The microcapsules obtained at the end of the second step in comparative examples 8, 9 and 10 were tested in a type II dissolutest in accordance with the European Pharmacopoeia 4th edition, at 37° C. and with stirring at 100 rpm, in the following media:
HCl at pH 1.4,
HCl at pH 1.4 for 2 hours and then KH₂PO₄/NaOH buffer medium, at pH 6.8.

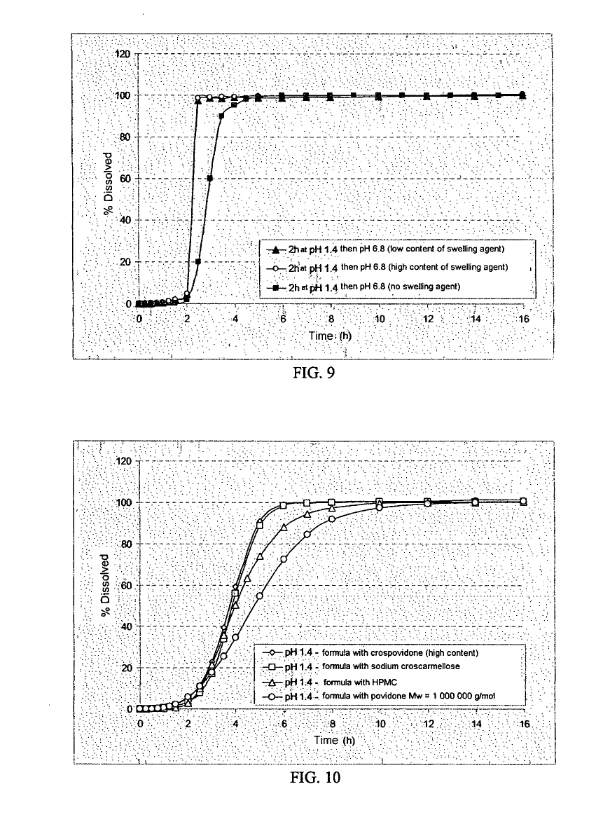

The dissolution profiles of examples 8, 9 and 10 are presented in FIGS. 8 and 9. It is noted that:
a broad kinetics range can be obtained at pH 1.4 depending on the amount of swelling agent incorporated into the formulation;
the release at pH 6.8 remains rapid regardless of the composition under consideration.

### Example 11

### Preparation of Microcapsules of Acyclovir Containing a Swelling Agent (Sodium Croscarmellose)

### Step 1:

300 g of acyclovir, 50 g of low molar mass hydroxypropylcellulose (Klucel® EF (Hercules)) and 150 g of sodium croscarmellose (Ac-Di-Sol®/FMC) are suspended in 1200 g of purified water. The solution is sprayed onto 500 g of neutral microspheres (NP Pharm) in a Glatt® GPCG1 spray coater.

### Step 2:

100 g of hydrogenated cottonseed oil (Penwest) and 100 g of poly(methacrylic acid) (ethyl acrylate) Eudragit® L100-55 (Röhm) are dissolved under hot conditions in ethanol. The solution is sprayed onto 750 g of previously prepared microparticles.

### Example 12

### Preparation of Microcapsules of Acyclovir Containing a Swelling Agent (Hydroxypropylmethylcellulose)

### Step 1:

300 g of acyclovir, 50 g of low molar mass hydroxypropylcellulose (Klucel® EF (Hercules)) and 150 g of hydroxypropylmethylcellulose (Pharmacoat 615/Shin-Etsu) are suspended in 1200 g of purified water. The solution is sprayed onto 500 g of neutral microspheres (NP Pharm) in a Glatt® GPCG1 spray coater.

100 g of hydrogenated cottonseed oil (Penwest), 100 g of poly(methacrylic acid) (ethyl acrylate) Eudragit® L100-55 (Röhm) and 50 g of poly(methacrylic acid) (methyl methacrylate) Eudragit® S100 (Röhm) are dissolved under hot conditions in ethanol. The solution is sprayed onto 750 g of previously prepared microparticles.

### Example 13

### Preparation of Microcapsules of Acyclovir Containing a Swelling Agent (Povidone of Molar Mass Mw=1 000 000 g/mol)

### Step 1:

350 g of acyclovir, 50 g of low molar mass hydroxypropylcellulose (Klucel® EF (Hercules)) and 100 g of high molar mass povidone (Kollidon® 90 (BASF)) are suspended in 1200 g of purified water. The solution is sprayed onto 500 g of neutral microspheres (NP Pharm) in a Glatt® GPCG1 spray coater.

### Step 2:

100 g of hydrogenated cottonseed oil (Penwest), 50 g of poly(methacrylic acid) (ethyl acrylate) Eudragit® L100-55 (Röhm) and 100 g of poly(methacrylic acid) (methyl methacrylate) Eudragit® S100 (Röhm) are dissolved under hot conditions in ethanol. The solution is sprayed onto 750 g of previously prepared microparticles.

The microcapsules obtained at the end of the second step in examples 10, 11, 12 and 13 were tested in a type II dissolutest in accordance with the European Pharmacopoeia 4th edition, at 37° C. and with stirring at 100 rpm, at pH 1.4.

The dissolution profiles are presented in FIG. 10.

### Example 14

### Preparation of Microcapsules of Acyclovir Containing a Swelling Agent (Granulation+Spray-Coating)

### Step 1:

700 g of acyclovir, 50 g of povidone (Plasdone®/ISP) and 250 g of crospovidone (Polyplasdone®/ISP) are dry-mixed beforehand in a laboratory granulator (Lodige) for 5 minutes. This pulverulent mixture is then granulated with water (200 g). The granules are dried at 40° C. in a ventilated oven, and then calibrated on a 500 μm screen. The 200-500 μm fraction is selected by screening.

### Step 2:

100 g of hydrogenated palm oil (Huls), 100 g of poly(methacrylic acid) (ethyl acrylate) Acrycoat L100D and 50 g of poly(methacrylic acid) (methyl methacrylate) Acrycoat® S100 (NP Pharm) are dissolved under hot conditions in isopropanol. The solution is sprayed onto 750 g of previously prepared microparticles.

The microcapsules obtained at the end of the second step of example 13 were tested in a type II dissolutest in accordance with the European Pharmacopoeia 4th edition, at 37° C. and with stirring at 100 rpm, in the following media:
HCl at pH 1.4,
HCl at pH 1.4 for 2 hours and then KH₂PO₄/NaOH buffer medium, at pH 6.8.

The dissolution profiles are presented in FIG. 11.

### Example 15

### Preparation of Microcapsules of Acyclovir Containing a Swelling Agent (Extrusion/Spheronization+Spray-Coating)

### Step 1:

700 g of acyclovir, 50 g of povidone (Plasdone®/ISP) and 250 g of crospovidone (Kollidon® CL/BASF) are premixed with 150 g of water in a laboratory mixer (Kitchen-Aid) for 5 minutes. This pasty mixture is extruded through a 0.5 mm screen using an extruder 20 (Caleva). The filaments obtained are then spheronized using a spheronizer 250 (Caleva). The particles obtained are dried at 40° C. in a fluidized airbed. The 300-700 μm fraction is selected by screening.

### Step 2:

100 g of hydrogenated palm oil (Huls), 100 g of poly(methacrylic acid) (ethyl acrylate) Acrycoat L100D and 50 g of poly(methacrylic acid) (methyl methacrylate) Acrycoat® S100 (NP Pharm) are dissolved under hot conditions in isopropanol. The solution is sprayed onto 750 g of previously prepared microparticles.

The microcapsules obtained at the end of the second step of example 14 were tested in a type II dissolutest in accordance with the Pharmacopoeia 4th edition, at 37° C. and with stirring at 100 rpm, in the following media:
HCl at pH 1.4,
HCl at pH 1.4 for 2 hours and then KH₂PO₄/NaOH buffer medium, at pH 6.8.

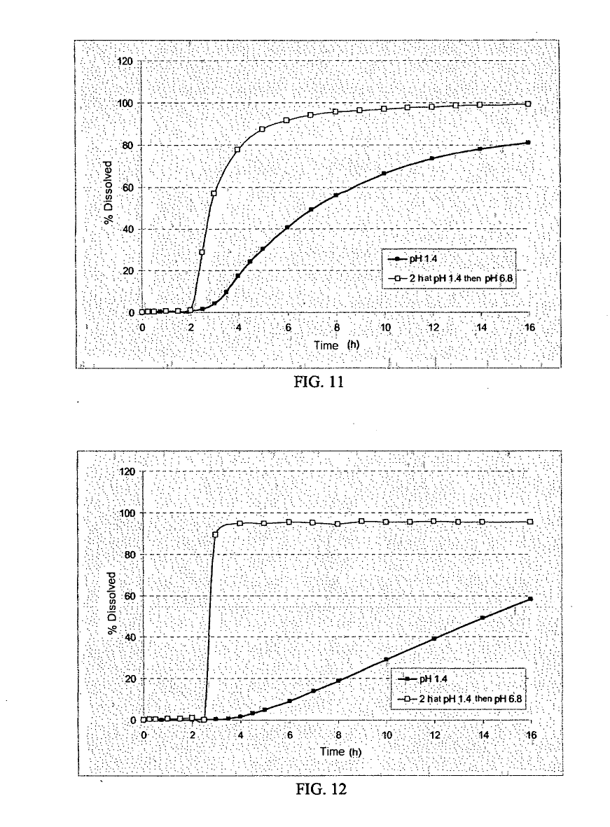

The dissolution profiles are presented in FIG. 12.

### Example 16

### Preparation of Microcapsules of Acyclovir Containing a Swelling Agent (Compacting+Spray-Coating)

### Step 1:

590 g of acyclovir, 10 g of magnesium stearate and 400 g of crospovidone are mixed using a laboratory mixer (Kitchen-Aid type) for 5 minutes. This mixture is then compacted using an Alexanderwerk WP120 laboratory compactor. The product obtained is then granulated using an Erweka oscillating granulator equipped with a 500 μm screen. The 100-500 μm fraction of the product obtained is selected by screening.

### Step 2:

100 g of hydrogenated palm oil (Huls), 100 g of poly(methacrylic acid) (ethyl acrylate) Acrycoat L100D and 50 g of poly(methacrylic acid) (methyl methacrylate) Acrycoat® S100 (NP Pharm) are dissolved under hot conditions in isopropanol. The solution is sprayed onto 750 g of previously prepared microparticles.

The microcapsules obtained at the end of the second step of example 14 were tested in a type II dissolutest in accordance with the Pharmacopoeia 4th edition, at 37° C. and with stirring at 100 rpm, in the following media:
HCl at pH 1.4,
HCl at pH 1.4 for 2 hours and then KH₂PO₄/NaOH buffer medium, at pH 6.8.

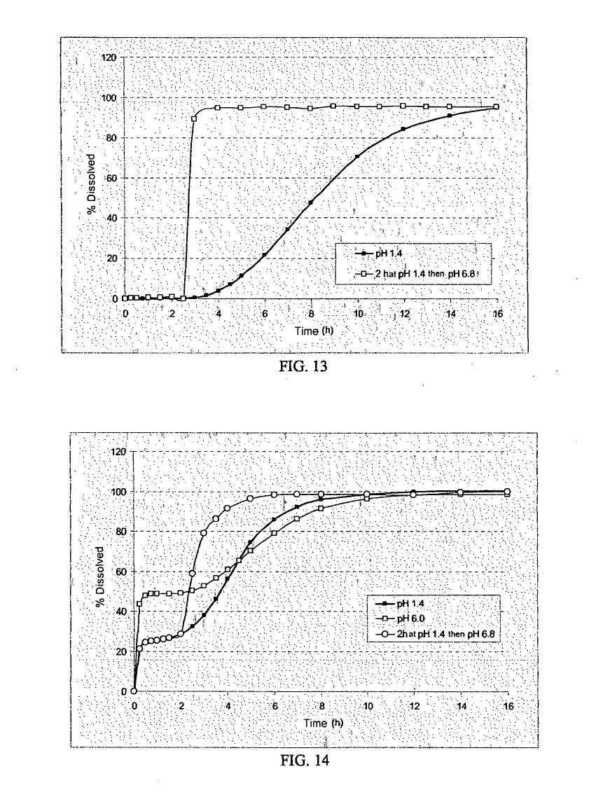

The dissolution profiles are presented in FIG. 13.

### Example 17

### Mixture of Microunits Having Various Release Profiles

Various microunits of acyclovir are prepared, in which microunits:
25% by weight of the acyclovir is in the form of immediate-release microunits as obtained at the end of the first step of example 12,
25% by weight of the acyclovir is in the form of delayed- and prolonged-release microunits as obtained at the end of the second step of example 10, and
50% by weight of the acyclovir is in the form of delayed- and prolonged-release microcapsules as obtained at the end of the second step of example 12.

The microcapsules of example No. 10 begin to rapidly release their content above pH>5.5 (use of Eudragit® L100-55).

The microcapsules of example No. 12 begin to rapidly release their content above pH>6.5 (use of 67% Eudragit® L100-55 and 33% Eudragit® S100).

The profiles are presented in FIG. 14 which show that various release phases are obtained, thereby optimizing the release of an active principle in front of its absorption window.

What is claimed is:

1. An oral medicament comprising a plurality of microcapsules for the modified release of active principle(s), at least some of said microcapsules consisting individually of a microparticle comprising at least one active principle, in particular at least one low-solubility active principle (with the exclusion of carvedilol), coated with at least one coating for the modified release of the active principle(s), said release being controlled by two distinct trigger mechanisms, one being based on a variation in pH and the other allowing the release of the active principle(s) after a predetermined period of time spent in the stomach,
said coating also conferring on the microcapsules an in vitro dissolution behavior such that:
at constant pH 1.4, the dissolution profile comprises a lag phase less than or equal to 7 hours, preferably less than or equal to 5 hours, and even more preferably between 1 and 5 hours, in duration;
the passing from pH 1.4 to pH 7.0 results in a release phase which begins without any lag time;
characterized
in that at least some of said microcapsules comprise at least one swelling agent,
and in that the fraction by weight of the active principle(s) released during the lag phase is less than or equal to 15% by weight per hour, preferably less than or equal to 10% by weight per hour, and even more preferably less than or equal to 5% by weight per hour.

2. The medicament as claimed in claim 1, characterized in that the swelling agent comprises at least one hydrophilic pharmaceutically acceptable compound which exhibits a degree of swelling in water at 25° C. of greater than or equal to 10% by weight, preferably greater than or equal to 15% by weight, and even more preferably greater than or equal to 20%.

3. The medicament as claimed in claim 1, characterized in that the swelling agent is chosen from those which allow the microcapsules to release at least 50% by weight of the active principle, after 16 h at pH 1.4, in an in vitro dissolution test.

4. The medicament as claimed in claim 2, characterized in that the swelling agent is in the form of microparticles which have an average diameter of between 5 and 200 μm, and preferably between 10 and 50 μm.

5. The medicament as claimed in claim 2, characterized in that the concentration (Cd) of swelling agent is defined as follows (in % by weight relative to the total mass of the microcapsules):
3≦Cd≦40,
preferably, 4≦Cd≦30,
and even more preferably, 5≦Cd≦25.

6. The medicament as claimed in claim 2 or 3, characterized in that the swelling agent is chosen from the group of following products:
crosslinked polyvinylpyrrolidones (e.g. polyplasdone or crospovidone),
crosslinked carboxyalkylcelluloses: crosslinked carboxymethylcelluloses (e.g. crosslinked sodium croscarmellose),
and also high molar mass hydrophilic polymers (greater than or equal to 100 000 D) such as:
polyvinylpyrrolidones,
polyalkylene oxides (e.g. polyethylene oxide or polypropylene oxide),
(hydroxy)(alkyl)celluloses (e.g. hydroxypropylcellulose, hydroxypropylmethylcellulose),
carboxyalkylcelluloses (e.g. carboxymethyl-cellulose),
celluloses (powder or microcrystalline),
modified starches (for example modified with sodium glycolate),
natural starches (for example from maize, wheat or potato),
sodium alginate,
potassium polacriline,
and mixtures thereof.

7. The medicament as claimed in one of the preceding claims, characterized in that it comprises at least one wetting agent, preferably chosen from the group of following products:
anionic surfactants, preferably in the subgroup of alkali metal or alkaline earth metal salts of fatty acids, stearic acid and/or oleic acid being preferred,
and/or nonionic surfactants, preferably in the following subgroup:
polyoxyethylenated oils, preferably polyoxyethylenated hydrogenated castor oil,
polyoxyethylene-polyoxypropylene copolymers,
polyoxyethylenated sorbitan esters,
polyoxyethylenated castor oil derivatives,
stearates, preferably calcium stearate, magnesium stearate, aluminum stearate or zinc stearate,
stearyl fumarates, preferably sodium stearyl fumarate,
glyceryl behenate,
and mixtures thereof.

8. The medicament as claimed in one of the preceding claims, characterized in that the swelling agent and/or the wetting agent is contained in the microparticle of active principle.

9. The medicament as claimed in one of the preceding claims, characterized in that the microcapsules of active principle(s) that it comprises are capable of releasing at least 80% by weight of the active principle(s), after 12 h at pH=7.0, in an in vitro dissolution test.

10. The medicament as claimed in one of the preceding claims, characterized in that at least some of the microcapsules for the modified release of active principle(s) each comprise:
a microparticle of active principle(s), coated with
at least one coating for the modified release of the active principle(s).

11. The medicament as claimed in one of the preceding claims, characterized in that at least some of said microcapsules for the modified release of active principle comprise:
a neutral core,
at least one active layer comprising the active principle(s) and coating the neutral core, and
at least one coating for the modified release of the active principle.

12. The medicament as claimed in one of the preceding claims, characterized in that:
the coating for the modified release of the active principle(s) comprises a composite material
comprising:
at least one hydrophilic polymer A bearing groups that are ionized at neutral pH,
at least one hydrophobic compound B,
representing a mass fraction (% weight relative to
the total mass of the microcapsules)≦40; and
the microcapsules have an average diameter of less than 2000 μm.

13. The medicament as claimed in claim 12, characterized in that the composite material AB of the coating for the modified release of the low-solubility active principle is such that:
the weight ratio B/A is between 0.2 and 1.5, preferably between 0.5 and 1.0,
and the hydrophobic compound B is selected from products that are crystalline in the solid state and have a melting point MpB≧40° C., preferably MpB≧50° C., and even more preferably 40° C.≦MpB≦90° C.

14. The medicament as claimed in either of claims 12 and 13, characterized in that the hydrophilic polymer A is chosen from:
A.a copolymers of (meth)acrylic acid and of a (meth)acrylic acid alkyl ester, and mixtures thereof;
A.b cellulose derivatives, preferably cellulose acetates, cellulose phthalates, cellulose succinates and mixtures thereof, and even more preferably hydroxypropylmethylcellulose phthalates, hydroxypropylmethylcellulose acetates, hydroxypropylmethylcellulose succinates and mixtures thereof;
and mixtures thereof.

15. The medicament as claimed in one of claims 12 to 14, characterized in that the compound B is chosen from the group of following products:
B.a plant waxes taken on their own or as mixtures with one another;
B.b hydrogenated plant oils taken on their own or as mixtures with one another;
B.c mono- and/or di- and/or triesters of glycerol and of at least one fatty acid;
B.d mixtures of monoesters, of diesters and of triesters of glycerol and of at least one fatty acid;
B.e and mixtures thereof.

16. The medicament as claimed in claim 15, characterized in that the compound B is chosen from the group of following products:
hydrogenated cottonseed oil, hydrogenated soya bean oil, hydrogenated palm oil, glyceryl behenate, hydrogenated castor oil, tristearin, tripalmitin, trimyristin, yellow wax, hard fat or fat that can be used as bases for suppositories, anhydrous dairy fats, lanolin, glyceryl palmitostearate, glyceryl stearate, lauryl macrogolglycerides, cetyl alcohol, polyglyceryl diisostearate, diethylene glycol monostearate, ethylene glycol monostearate, Omega 3 and any mixture thereof,
preferably from the subgroup of following products: hydrogenated cottonseed oil, hydrogenated soya bean oil, hydrogenated palm oil, glyceryl behenate, hydrogenated castor oil, tristearin, tripalmitin, trimyristin, and any mixture thereof.

17. The medicament as claimed in claim 16, characterized in that the compound B is chosen:
from the group of products sold under the following trademarks: Dynasan®, Cutina®, Hydrobase®, Dub®, Castorwax®, Croduret®, Compritol®, Sterotex®, Lubritab®, Apifil®, Akofine®, Softtisan®, Hydrocote®, Livopol®, Super Hartolan®, MGLA®, Corona®, Protalan®, Akosoft®, Akosol®, Cremao®, Massupol®, Novata®, Suppocire®, Wecobee®, Witepsol®, Lanolin®, Incromega®, Estaram®, Suppoweiss®, Gelucire®, Precirol®, Emulcire®, Plurol diisostearique®, Geleol®, Hydrine® and Monthyle® and mixtures thereof;
and also from the group of additives for which the codes are the following: E 901, E 907, E 903 and mixtures thereof;
and, preferably from the group of commercial products sold under the following trademarks: Dynasan® P60, Dynasan® 114, Dynasan® 116, Dynasan® 118, Cutina® HR, Hydrobase® 66-68, Dub® HPH, Compritol® 888, Sterotex® NF, Sterotex® K, Lubritab® and mixtures thereof.

18. The medicament as claimed in any one of claims 12 to 17, characterized in that the coating of the microcapsules for the modified release of active principle comprises a single coating film comprising the composite AB.

19. An oral medicament comprising a plurality of microcapsules for the modified release of active principle(s), at least some of said microcapsules consisting individually of a microparticle comprising at least one active principle, in particular at least one low-solubility active principle (with the exclusion of carvedilol), coated with at least one coating for the modified release of the active principle(s), said release being controlled by two distinct trigger mechanisms, one based on a variation in pH and the other allowing the release of the active principle(s) after a predetermined period of time spent in the stomach,
said coating:
also conferring on the microcapsules an in vitro dissolution behavior such that:
at constant pH 1.4, the dissolution profile comprises a lag phase of less than or equal to 7 hours, preferably less than or equal to 5 hours, and even more preferably between 1 and 5 hours, in duration;
the passing from pH 1.4 to pH 7.0 results in a release phase which begins without any lag time;
and comprising a composite material comprising at least one hydrophilic polymer A bearing groups that are ionized at neutral pH and at least one hydrophobic compound B;
characterized in that at least some of said microcapsules comprise at least one release helper capable of increasing the permeability of the coating for the modified release of the active principle(s), and in that the fraction by weight of the active principle(s) released during the lag phase is less than or equal to 15% by weight per hour, preferably less than or equal to 10% by weight per hour, and even more preferably less than or equal to 5% by weight per hour.

20. The medicament as claimed in claim 19, characterized in that the release helper consists of at least one swelling agent.

21. The medicament as claimed in claim 19 or 20, characterized in that the coating of the microcapsules confers on them an in vitro dissolution behavior such that at least 50% by weight of the active principle(s) is released after 16 h at pH 1.4.

22. The medicament as claimed in one of claims 1 to 20, characterized in that it comprises a mixture of various populations of microunits containing active principle(s), with the exclusion of carvedilol, these populations differing from one another by virtue of their respective in vitro dissolution profiles, for at least one pH value of between 1.4 and 7.4.

23. The medicament as claimed in claim 21 and at least one of the other preceding claims, characterized in that the microunits are microcapsules for the modified release of active principle(s) and, optionally, microunits for the immediate release of active principle(s).

24. The medicament as claimed in claim 22, characterized in that the populations of microcapsules for the modified release of active principle differ by virtue of their respective trigger pHs.

25. The medicament as claimed in claim 22, characterized in that the populations of microcapsules for the modified release of active principle differ by virtue of their respective trigger times.

26. The medicament as claimed in claim 22, characterized in that it comprises:
i. at least one population of microunits containing immediate-release active principle;
ii. at least one population P1 of microcapsules for the modified release of active principle(s), and
iii. at least one population P2 of microcapsules for the modified release of active principle(s);
and in that the respective trigger pHs of P1 and of P2 differ by at least 0.5 pH unit, preferably by at least 0.8 pH unit, and even more preferably by at least 0.9 pH unit.

27. The medicament as claimed in claim 22, characterized in that the respective trigger pHs of the various populations of microcapsules for the modified release of active principle(s) are between 5 and 7.

28. The medicament as claimed in claim 22, characterized in that it comprises:
i. at least one population of microunits containing immediate-release active principle(s),
ii. at least one population P1′ of microunits containing active principle(s) made up of microcapsules for the modified release of the active principle(s), the trigger pH of which is equal to 5.5, and
iii. at least one population P2′ of microunits containing active principle(s) made up of microcapsules for the modified release of the active principle(s), the trigger pH of which is equal to 6.0 or 6.5.

29. The medicament as claimed in one of claims 22 to 28, characterized in that the release profile, measured in an in vitro release test, is as indicated hereinafter:
less than 20% of the active principle(s) is released after 2 hours at pH=1.4;
at least 50% of the active principle(s) is released after 16 hours at pH=1.4.

30. The medicament as claimed in one of claims 22 to 29, characterized in that it comprises at least one population of microunits containing immediate-release active principle(s), the behavior of which in an in vitro dissolution test is such that at least 80% of the active principle(s) is released in 1 hour at any pH between 1.4 and 7.4.

31. The medicament as claimed in one of claims 22 to 30, characterized in that the proportion of low-solubility active principle(s) in the microunits containing active principle(s) (expressed as % by weight on a dry weight basis, relative to the total mass of the microunits) is between 5 and 80, preferably between 10 and 70, and even more preferably between 15 and 60.

32. The medicament as claimed in one of claims 22 to 31, characterized in that the microunits containing immediate-release active principle(s) are uncoated microparticles.

33. The medicament as claimed in one of the preceding claims, characterized in that it is in the form of a single daily oral dose comprising from 5000 to 500 000 microunits containing active principle(s).

34. The medicament as claimed in one of the preceding claims, characterized in that it is in the form of a single daily oral dose comprising from 5000 to 500 000 microcapsules for the modified release of active principle(s).

35. The medicament as claimed in one of the preceding claims, characterized in that it is in the form of a sachet of microcapsule powder, of a liquid suspension of microcapsules, of a tablet obtained from microcapsules, or of a gelatin capsule containing microcapsules.

36. The medicament as claimed in one of the preceding claims, characterized in that the active principle(s) can be chosen from at least one of the following main varieties of active substances, e.g.: antiulcer agents, antidiabetic agents, anticoagulants, antithrombotics, blood-lipid-lowering agents, antiarrhythmics, vasodilators, anti-angina agents, antihypertensives, vasoprotectors, fertility promoters, inhibitors and inducers of uterine labor, contraceptives, antibiotics, antifungals, antivirals, anticancer agents, anti-inflammatories, analgesics, antiepileptics, anti-parkinsonian agents, neuroleptics, hypnotics, anxiolytics, psychostimulants, antimigraine agents, antidepressants, antitussives, antihistamines or anti-allergic agents, agents for combating congestive heart failure, angina pectoris, left ventricular hypertrophy, cardiac arrhythmias, myocardial infarction, reflex tachycardia, ischemic heart disease, atheromatosis, diabetes mellitus-related hypertension, portal hypertension, vertigo, abradycardia, arterial hypotension, water and sodium retention, acute renal insufficiency, orthostatic hypotension and cerebral congestion, and mixtures thereof.

37. The medicament as claimed in one of the preceding claims, characterized in that the active principle(s) is (are) chosen from the group of products comprising: acetylsalicylic acid, carbamazepine pentoxifylline, prazosine, acyclovir, nifedipine, diltiazem, naproxen, ibuprofen, flurbiprofen, ketoprofen, fenoprofen, indomethacin, diclofenac, fentiazac, estradiol valerate, metoprolol, sulpiride, captopril, cimetidine, zidovudine, nicardipine, terfenadine, atenolol, salbutamol, carbamazepine, ranitidine, enalapril, simvastatin, fluoxetine, alprazolam, famotidine, ganciclovir, famciclovir, spironolactone, 5-asa, quinidine, perindopril, morphine, pentazocine, paracetamol, omeprazole, lansoprazole, metoclopramide, aminosalicylic acid, nalidixic acid, amoxicillin, amoxicillin and potassium clavulanate, ampicillin, ampicillin and sulbactam, azithromycin, bacampicillin, carbenicillin-indanyl-sodium (and other carbenicillin salts), capreomycin, cefadroxil, cefazoline, cephalexine, cephalothine, cephapirine, cephacelor, cephprozile, cephadrine, cefamandole, cefonicide, ceforanide, cefuroxime, cefixime, cefoperazone, cefotaxime, cefpodoxime, ceftaxidime, ceftibuten, ceftizoxime, ceftriaxone, cefepime, cefinetazole, cefotetan, cefoxitin, ciprofloxacin, clarithromycin, clindamycin, clofazimine, cloxacillin, cotriamoxazole, cycloserine, dicloxacillin, dirithromycin, erythromycin (and erythromycin salts such as estolate, ethyl succinate, gluceptate, lactobionate, stearate), ethambutol-HCl and other salts, ethionamide, fosfomycin, imipenem, isoniazide, levofloxacin, lomefloxacin, loracarbef, methicillin, methenamine, metronidazole, metoclopramide, mezlocillin, nafcillin, nitrofurantoin, norfloxacine, novobiocine, ofloxacine, oxacillin, penicillin V, penicillin salts, penicillin complexes, pentamidine, piperacillin, piperacillin and tazobactam, sparfloxacine, sulfacytine, sulfamerazine, sulfamethazine, sulfamethixole, sulfasalazine, sulfisoxazole, sulfapyrizine, sulfadiazine, sulfinethoxazole, sulfapyridine, ticarcillin, ticarcillin and potassium clavulanate, trimethoprim, trimetrexate, troleanomycin, vancomycin, verapamil and mixtures thereof.

38. The medicament as claimed in one of the preceding claims, characterized in that the active principle(s) is (are) a low-solubility active principle or low-solubility active principles.

39. The use of the microcapsules for the modified release of active principle(s) (with the exclusion of carvedilol) as defined in any one of claims 1 to 38 and, optionally, of the microunits containing immediate-release active principle, for the preparation of pharmaceutical or dietetic, microparticulate oral galenic forms, preferably in the form of advantageously orodispersible tablets, of powders or of gelatin capsules.

40. The microcapsule as defined in any one of the preceding claims.
